(19) United States
(12) Patent Application Publication
(10) Pub. No.: US 2024/0009128 A1
(43) Pub. Date: Jan. 11, 2024

(54) METHODS AND SYSTEMS FOR USE OF LOW-VOLATILITY SOLVENTS FOR SPRAY DRYING

(71) Applicant: Serán BioScience, LLC, Bend, OR (US)

(72) Inventors: Mark Kastantin, Bend, OR (US); Erica Schlesinger, Sisters, OR (US); Dan Smithey, Bend, OR (US)

(21) Appl. No.: 18/348,269

(22) Filed: Jul. 6, 2023

Related U.S. Application Data
(60) Provisional application No. 63/367,898, filed on Jul. 7, 2022.

(51) Int. Cl. A61K 9/16; F26B 3/00

(52) U.S. Cl. CPC A61K 9/1688; F26B 3/00

(57) ABSTRACT

Pharmaceutical formulations, methods for spray drying pharmaceutical formations, and systems for spray drying pharmaceutical formulation are provided. In one example, a method for spray drying a pharmaceutical formulation may include selecting a solvent mixture comprising a low volatility solvent based on a solubility of a drug of the pharmaceutical formulation in the solvent mixture, forming a solution of the drug of the pharmaceutical formulation in the solvent mixture, and spray drying the solution using a spray drying mass ratio that is selected based on a relationship between a glass transition temperature of the pharmaceutical formulation and a relative saturation of the low volatility solvent during the spray drying, the spray drying mass ratio defining a liquid feed rate of the solution to a gas feed rate of a drying gas, and using a process outlet temperature that is less than the glass transition temperature of the pharmaceutical formulation.

### CROSS REFERENCE TO RELATED APPLICATIONS

The present application claims priority to U.S. Provisional Application No. 63/367,898 entitled “METHODS AND SYSTEMS FOR USE OF LOW-VOLATILITY SOLVENTS FOR SPRAY DRYING” filed Jul. 7, 2022. The entire contents of the above identified application is hereby incorporated by reference for all purposes.

### FIELD

The present description relates generally to preparing a pharmaceutical formulation using low volatility processing solvents for spray drying.

### BACKGROUND/SUMMARY

Spray drying is a technique commonly used in the production of pharmaceuticals. For example, spray drying may be used to produce a powder with a defined particle size distribution for downstream processing, as an efficient way to remove solvent, and/or to create amorphous solid dispersions to increase a bio-performance of a pharmaceutical compound (e.g., a drug). In many cases, certain materials or excipients are combined with the pharmaceutical compounds in the solvents. However, many pharmaceutical compounds have poor solubility in the organic solvents typically used for spray drying.

Common spray drying solvents, or mixtures thereof, typically have boiling points of 100° C. or lower (e.g., acetone, methanol, ethanol, isopropanol, ethyl acetate, acetonitrile, tetrahydrofuran, methylene chloride, water, etc.). A low boiling point solvent or solvent system is advantageous for spray drying because higher boiling points typically warrant higher process temperatures during spray drying, which can lead to poor compound chemical and/or physical stability, equipment complexity, or other process challenges. However, despite the advantageously low boiling point, the low solubility of some pharmaceutical compounds in these low boiling point solvents dramatically reduces manufacturing throughput of formulations (e.g., amorphous dispersions) containing these compounds.

Very often, the solubility of these compounds is significantly higher in solvents like dimethylsulfoxide (DMSO), n-methyl-2-pyrrolidone (NMP), dimethylformamide (DMF), and n,n-dimethylacetamide (DMAC). However, these solvents have low volatility, as evidenced by their high boiling points (e.g., greater than 150° C.) and are typically avoided for use in spray drying. Spray drying with these solvents typically leads to high processing temperatures that may degrade the pharmaceutical compound or excipients, or high levels of residual solvent may remain in the spray-dried material that may be challenging to remove to acceptable levels for in vivo use.

In one example, the issues described above may be at least partially addressed by a pharmaceutical formulation, a method for preparing the pharmaceutical formation, and a system for preparing the pharmaceutical formulation. The method for preparing the pharmaceutical formulation may include selecting a solvent mixture comprising a first percentage of a low volatility solvent and a second percentage of a high volatility solvent based on a solubility of a drug of the pharmaceutical formulation in the solvent mixture, forming a solution of the drug of the pharmaceutical formulation in the solvent mixture, and spray drying the solution to form the dispersion using a spray drying mass ratio that is selected based on a relationship between a glass transition temperature of the pharmaceutical formulation and a relative saturation of the low volatility solvent during the spray drying, the spray drying mass ratio defining a liquid feed rate of the solution to a gas feed rate of a drying gas during the spray drying, and using an outlet temperature that is less than the glass transition temperature of the pharmaceutical formulation. In this way, the solubility of the pharmaceutical compound may be enhanced during the spray drying process while increasing processing throughput and decreasing temperature-related issues.

It should be understood that the summary above is provided to introduce in simplified form a selection of concepts that are further described in the detailed description. It is not meant to identify key or essential features of the claimed subject matter, the scope of which is defined uniquely by the claims that follow the detailed description. Furthermore, the claimed subject matter is not limited to implementations that solve any disadvantages noted above or in any part of this disclosure.

### BRIEF DESCRIPTION OF THE DRAWINGS

FIG. 1 illustrates a block diagram of a workflow for spray drying compounds using a blend of high volatility and low volatility solvents.

FIG. 2 shows a process map relating a process outlet temperature to a spray drying mass ratio.

FIG. 3 shows a first exemplary yield plot as a function of a difference between a glass transition temperature and a process outlet temperature for spray drying product out of solvent blends comprising a low volatility solvent.

FIG. 4 shows a second exemplary yield plot as a function of a difference between a glass transition temperature and a process outlet temperature for spray drying product out of solvent blends comprising a low volatility solvent.

FIG. 5 shows a dynamic vapor sorption plot relating DMSO uptake in a material as a function of DMSO relative saturation.

FIG. 6 shows a plot relating DMSO relative saturation to a glass transition temperature of a material.

FIG. 7 is a flow chart of an example method for optimizing spray drying parameters for spray drying with at least one low volatility solvent.

FIG. 8 is a flow chart of an example method for using a low volatility solvent as a processing solvent for spray drying a pharmaceutical formulation.

FIG. 9 shows an exemplary process map relating processing conditions to overall desirability with respect to throughput and yield.

FIG. 10 shows a graph of DMSO, DMAC, and DMF vapor pressures relative to vapor pressure of DMSO as a function of temperature.

### DETAILED DESCRIPTION

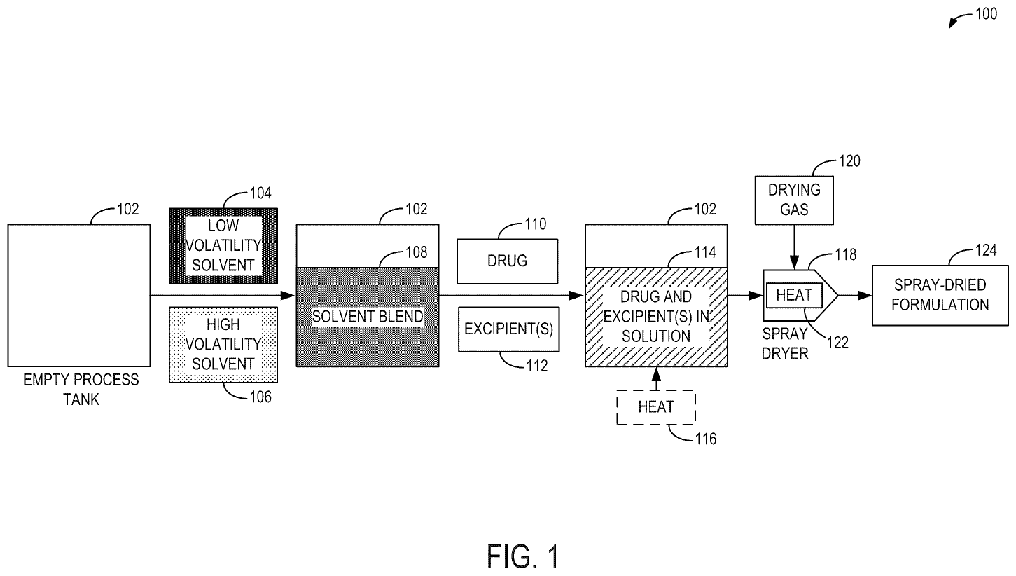

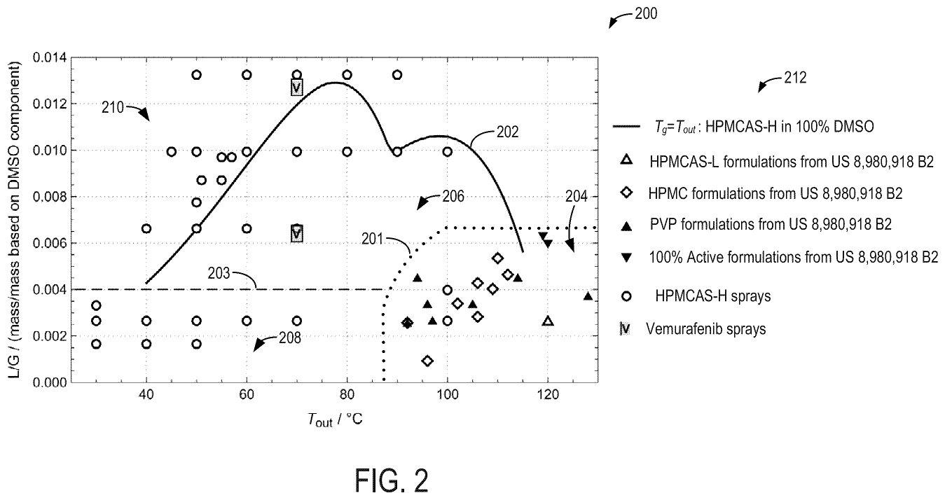

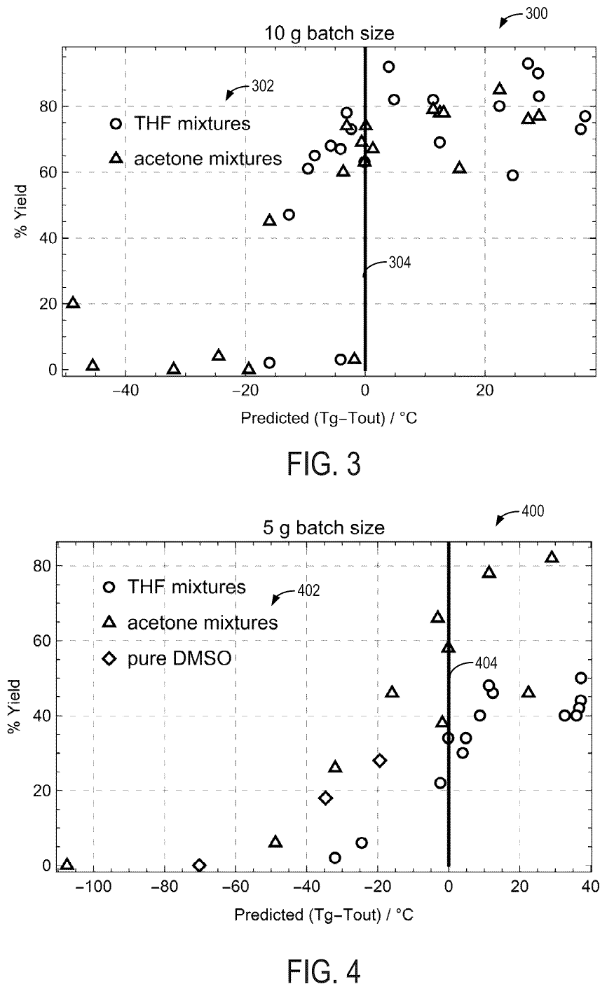

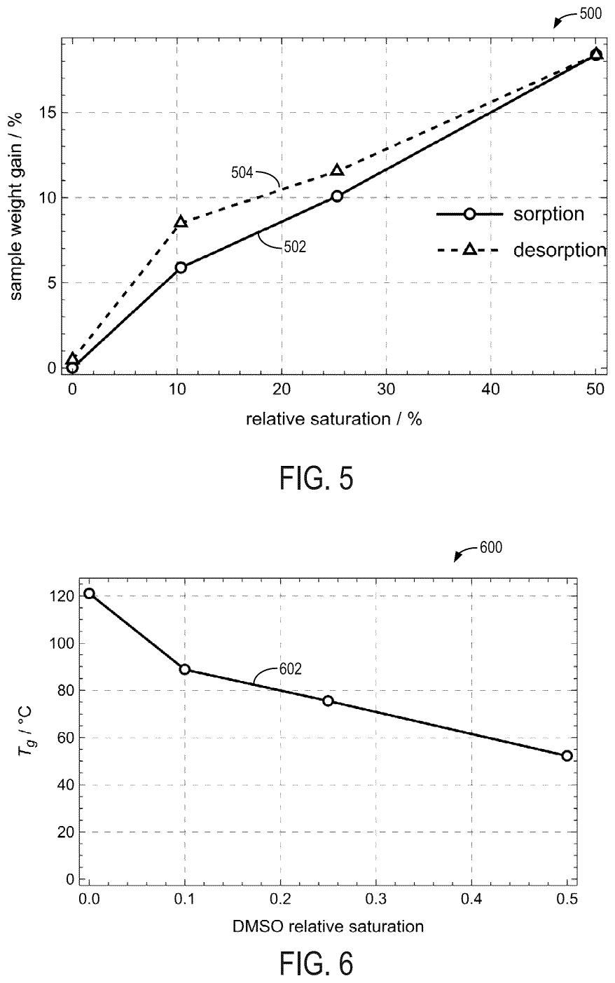

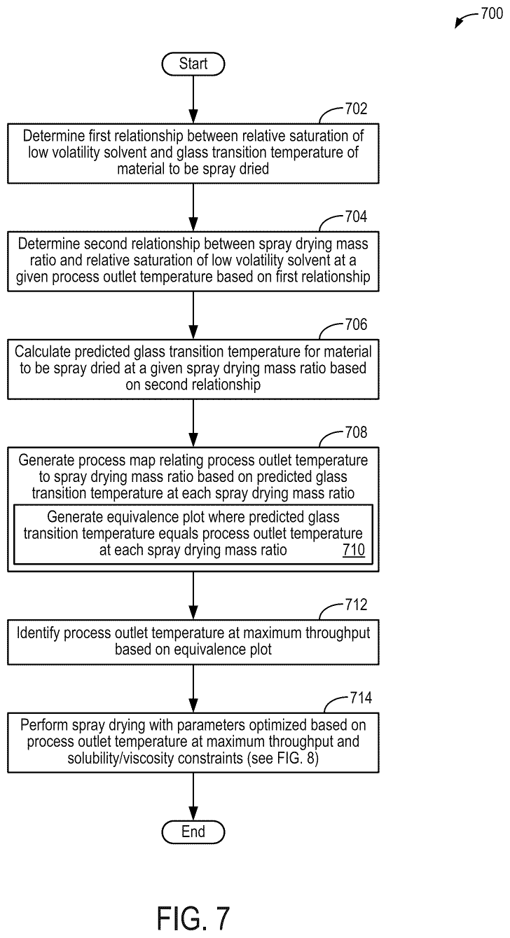

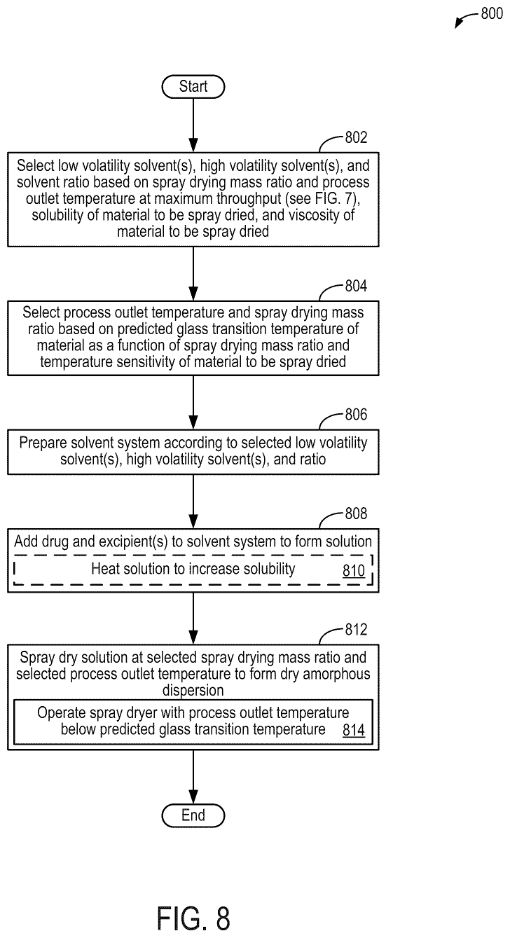

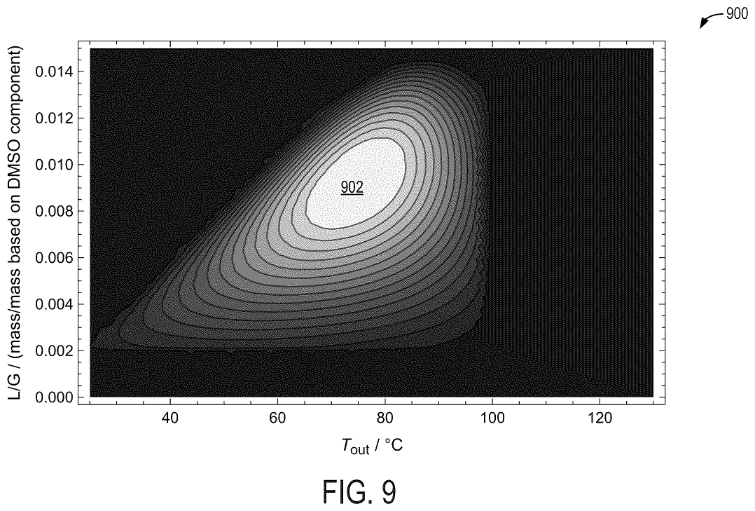

The following description relates to systems and methods for processing pharmaceutical compounds, also referred to herein as drugs, using low volatility solvents. The low volatility solvents may be combined with one or more high volatility solvents to bring the pharmaceutical compound into solution with excipient(s) prior to spray drying, such as according to the workflow illustrated in FIG. 1. The resulting spray-dried dispersion of the pharmaceutical compound and the excipient(s) may be referred to herein as a pharmaceutical formulation. As used herein, the term “low volatility solvent” refers to a solvent having a boiling point of at least 110° C., whereas the term “high volatility solvent” refers to a solvent having a boiling point that is less than 110° C. (e.g., 100° C. or lower). For example, the low volatility solvent(s) may be mixed with the high volatility solvent(s) in a ratio that is selected to optimize processing throughput, yield, and temperature, such as according to the methods of FIGS. 7 and 8. The processing parameters may be defined by a process map, such as shown in FIG. 2, and by defining a process outlet temperature that equals a glass transition temperature of the material being spray dried at each spray drying mass ratio. Herein, spray drying mass ratio refers to a ratio of mass of liquid to mass of drying gas fed into the spray dryer. Spray drying mass ratio may also be referred to as an L/G ratio as described further below. For example, yields may decrease when the process outlet temperature is greater than the glass transition temperature at the given spray drying mass ratio, such as demonstrated in FIGS. 3 and 4. For example, a relationship between a relative saturation of the low volatility solvent and the glass transition temperature may be determined by examining uptake of the solvent by the material at different relative saturations of the solvent and measuring the corresponding glass transition temperature, such as shown in FIGS. 5 and 6. FIG. 9 identifies various combinations of process conditions based on their desirability for increasing both throughput and yield. In this way, solubility of the pharmaceutical compound in the spray drying solvent may be increased by including the low volatility solvent while avoiding high outlet process temperatures that may result in degradation, resulting in increased throughput and yields for compounds that have relatively low solubility in the high volatility solvents typically used during spray drying.

Turning now to FIG. 1, it shows a workflow 100 for processing a pharmaceutical compound (e.g., a drug) by spray drying from a solution having a mixture of at least one low volatility solvent and at least one high volatility solvent. The workflow 100 includes a process tank 102 that is initially empty. A low volatility solvent 104 and a high volatility solvent 106 are each added to the process tank 102 to form a solvent blend 108. The low volatility solvent 104 may be selected from, for example, dimethylsulfoxide (DMSO), n-methyl-2-pyrrolidone (NMP), dimethylformamide (DMF), and n,n-dimethylacetamide (DMAC). In particular, DMSO may be selected as the low volatility solvent 104 due to its relatively high acceptable exposure limits, as residual low volatility solvent may remain after spray drying, as will be further described below. The high volatility solvent 106 may be selected from, for example, acetone, an alcohol (e.g., methanol, ethanol, isopropanol, n-propanol), acetonitrile (ACN), tetrahydrofuran (THF), methylene chloride (DCM), ethyl acetate (EtOAc), n-methyl tetrahydrofuran, chloroform, hexanes (e.g., n-hexane), acetic acid, cyclohexane, an ether (e.g., ethyl ether), and water. As will be elaborated with respect to FIG. 8, the particular solvents used as well as a ratio of the low volatility solvent 104 and the high volatility solvent 106 within the solvent blend 108 may be optimized to increase process throughput, increase yield, and decrease process temperatures. For example, the pharmaceutical compound may be more soluble in the low volatility solvent 104 than the high volatility solvent 106, and the low volatility solvent 104 may be more difficult to remove than the high volatility solvent 106 via spray drying. Further, in some examples, the low volatility solvent 104 and/or the high volatility solvent 106 may comprise more than one solvent. For example, the high volatility solvent 106 may be a blend of two or more high volatility solvents, such as a blend of methanol, acetone, and DCM in a selected ratio (e.g., percentage by weight or volume). Herein, a blend of solvents may refer to miscible mixture of solvents which may not be physically separated.

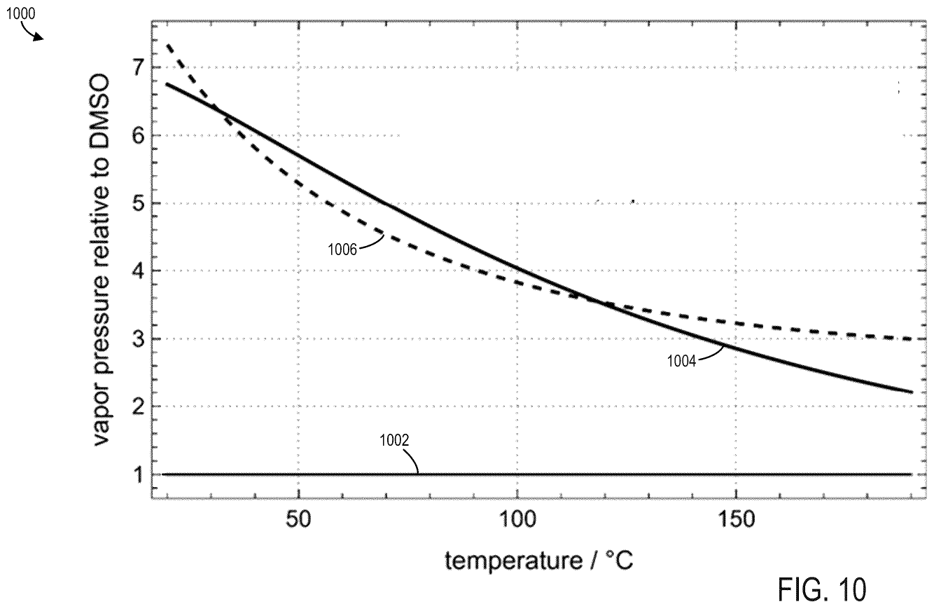

High volatility solvents and low volatility solvents are defined with respect to their boiling points at atmospheric pressure. However, it is recognized that vapor pressure of the solvent at temperatures used for spray drying is a more relevant parameter in determining a yield and quality of a spray dried dispersion. Generally, higher vapor pressures correspond to lower boiling points and vice versa. However, a magnitude of an effect of switching between two solvents with different boiling points may only be appreciated by comparing vapor pressures. Turning to FIG. 10 as an example, it shows a graph 1000 of vapor pressure relative to DMSO as a function of temperature. A plot 1002 corresponds to a relative vapor pressure of DMSO which is 1 at all temperatures where the y-axis is given relative to DMSO. A plot 1004 corresponds to vapor pressure of DMF relative to DMSO and a plot 1006 corresponds to vapor pressure of DMAC relative to DMSO. The boiling point of DMSO is 189° C. (462K), which is higher than the boiling point of DMF (153° C./426K) and DMAC (165° C./438K) but not by more than 10% on an absolute temperature scale. On the other hand, as shown in graph 1000, the vapor pressures of DMF and DMAC may be between 2× to 7× greater depending on the temperature. Effects of selecting DMSO as a low volatility solvent versus DMF or DMAC will be discussed further below.

Returning now to FIG. 1, a drug 110 and one or more excipients 112 are added to the solvent blend 108 in the process tank 102 to form a solution 114. For example, the drug 110 may be a crystalline form of the pharmaceutical compound that has relatively poor bioavailability, stability, and/or aqueous solubility, and the one or more excipients 112 may be polymers that, when blended with the drug 110, increase its bioavailability, stability, and/or aqueous solubility. For example, the one or more excipients 112 may include a saccharide, such as a derivative of cellulose (e.g., hydroxypropylcellulose, hydroxypropyl methylcellulose, hypromellose acetate succinate, etc.) or a sugar (e.g., xylitol, lactose, etc.), a protein (e.g., gelatin), and/or a synthetic polymer (e.g., methacrylic acid-methyl methacrylate copolymer or polyvinylpyrrolidone).

Although the workflow 100 shows the drug 110 and the one or more excipients 112 being added to the solvent blend 108, other orders of addition are also possible. For example, the drug 110 may be added to the low volatility solvent 104 to form a first solution comprising the drug 110 and the low volatility solvent 104. Then, the high volatility solvent 106 may be added to the first solution to form a second solution comprising the drug 110, the low volatility solvent 104, and the high volatility solvent 106. Further, the one or more excipients 112 may be added to the first solution or the second solution. As another example, the one or more excipients 112 may be added to the low volatility solvent 104, the high volatility solvent 106, or the solvent blend 108 at a same or different time relative to the drug 110 (e.g., before the drug 110, after the drug 110, or simultaneously with the drug 110). Further, when multiple excipients are included in the one or more excipients 112, each individual component of the one or more excipients 112 may be added to the solvent(s) at once or individually. Thus, the solution 114 may be formed via various orders of addition. Further, agitation may be used to enhance mixing within the process tank 102.

Optionally, heat 116 may be applied to the solution 114 to aid in the dissolution. For example, the solubility of the drug 110 in the solvent blend 108 may be higher at temperatures above ambient temperature. However, the solution 114 may be maintained below a temperature where the drug 110 or the one or more excipients 112 may start to degrade.

The solution 114 is fed from the process tank 102 to a spray dryer 118 configured to receive the solution 114 at a liquid feed rate (L). In general, as a concentration (e.g., percentage) of the low volatility solvent 104 in the solvent blend 108 increases, the liquid feed rate may decrease. The solution 114 may flow through an atomizing nozzle of the spray dryer 118, which breaks the liquid stream into a spray of droplets of a controlled size. The spray dryer 118 may also be configured to receive a drying gas 120 at a gas feed rate (G). A ratio of the liquid feed rate to the gas feed rate will be referred to herein as a L/G ratio. For example, the liquid feed rate may refer to a mass of the solution 114 processed per unit time, and the gas feed rate may be a mass of the drying gas 120 that flows through the spray dryer 118 per unit of time. Thus, the L/G ratio may express a ratio of the mass of the solution 114 to the mass of the drying gas 120 entering the spray dryer 118. Herein, L/G ratio may also be referred to as a spray drying mass ratio.

The drying gas 120 (which may be air or nitrogen, for example) may be heated to a drying gas inlet temperature via a heating element 122 of the spray dryer 118, and the heated drying gas 120 may contact the spray to evaporate the solvent blend 108 to form amorphous dispersions of the drug 110 and the one or more excipients 112, resulting in an output comprised of a spray-dried formulation 124. A temperature achieved at the outlet of the spray dryer 118 may be increased or decreased by adjusting the heating element 122 and thereby the drying gas inlet temperature. The temperature achieved at the outlet of the spray dryer 118 may be referred to herein as a process outlet temperature. As will be elaborated below, the process outlet temperature may be selected as a function of the L/G ratio. For example, a typical outlet temperature for the spray dryer 118 may range from 35-55° C. when pure acetone is used, but may range from 50-120° C. when a mixture of DMSO and acetone is used. Selection of the process outlet temperature will be further described below with respect to FIG. 2. In some examples, the spray-dried formulation 124 may undergo secondary drying. However, within the spray dryer 118 and during the secondary drying, the temperature may be kept lower than the boiling point of the low volatility solvent 104.

Solvent removal during the spray drying process is affected by both kinetic and thermodynamic properties, such as equilibrium partitioning of solvent between the vapor phase and the spray-dried formulation 124. The kinetic and thermodynamic properties are dependent on an identity of the low volatility solvent(s) 104 and high volatility solvent(s) 106 in the solvent blend 108 as well as their ratio and may create processing constraints for the spray drying process. The amount of the low volatility solvent(s) 104 in the solvent blend 108 may range from 0-100%. For example, the solvent blend 108 may include a first percentage of the low volatility solvent(s) 104 and a second, remaining percentage of the high volatility solvent(s) 106. As an example, the low volatility solvent(s) 104 may comprise at least 10% of the solvent blend 108 (e.g., by weight or by volume). However, as the percentage of the low volatility solvent(s) 104 in the solvent blend 108 increases, the process outlet temperature for the spray drying may increase and/or a process rate (e.g., the liquid feed rate) of the spray drying may decrease in order to aid the solvent removal.

A successful spray drying process uses process outlet temperatures (Tₒᵤₜ) that are below a glass transition temperature (T_{g}) of the spray-dried product (e.g., the spray-dried formulation 124 of FIG. 1), which comprises the active pharmaceutical ingredient (e.g., a drug), excipient(s), and residual solvent. If the process outlet temperature is above that of T_{g} of the spray-dried product, then the material will stick to equipment surfaces, resulting in unacceptably low yields. Moreover, a physical stability of the spray dried product may decrease dramatically when held at or above its glass transition temperature. The glass transition temperature of the spray-dried product laden with residual solvent will be termed a “wet T_{g}” as opposed to a “dry T_{g}” that is observed when all residual solvent has been removed from the product. The wet T_{g} is lower than the dry T_{g}.

When designing a spray drying process, higher process outlet temperatures result in a drier product because they reduce a relative saturation of solvent in the vapor stream when compared to a lower process outlet temperature given feeds at an equivalent L/G ratio. Thus, the wet T_{g} is expected to increase at higher process outlet temperatures because there is less residual solvent in the product. Because the thermodynamic principles involved in determining a magnitude of this effect are hard to predict a priori, it is not easily predicted whether a process outlet temperature increase of ΔT will change the wet T_{g} more or less than the magnitude of ΔT. Because of these competing effects, there are finite upper and lower thresholds on the process outlet temperature to ensure chemical and physical stability of the product. According to embodiments described herein, these upper and lower thresholds are functions of the L/G ratio in the spray dryer, and the relationship between these thresholds and the L/G ratio will depend on the formulation composition and solvent system. As used herein, the term “solvent system” may refer to a mixture of one or more solvents in a defined ratio or a single solvent. For example, the solvent system may comprise a solvent mixture (or solvent blend) comprising one or more low volatility solvents and/or one or more high volatility solvents, a single low volatility solvent, or a single high volatility solvent. For example, a solvent mixture is a solvent system comprising a plurality of solvents (e.g., a mixture of different solvents).

For example, turning briefly to FIG. 2, an example process map 200 is shown for spray drying pharmaceutical formulations from solvent blends using DMSO. The vertical axis of the process map 200 denotes the L/G ratio (e.g., calculated based on the mass fraction of DMSO in the solvent feed), and the horizontal axis represents the process outlet temperature Tₒᵤₜ. An increase in the L/G ratio corresponds to an increase in the spray drying throughput because more material is processed per unit of time. The process map 200 includes an equivalence plot 202 where the wet T_{g} is equal to the process outlet temperature for spray drying H-grade hypromellose acetate succinate (HPMCAS-H), an excipient, from 100% DMSO. The equivalence plot 202 may be empirically determined as a function of relative saturation of DMSO, as will be further described below with respect to FIG. 5.

The process map 200 may be generally divided into a plurality of regions, including a first region 204 bordered by a dotted line 201; a second region 206 bordered by a combination of the dotted line 201, a dashed line 203, and the equivalence plot 202; a third region 208 bordered by a combination of the dotted line 201 and the dashed line 203; and a fourth region 210 bordered by a combination of the equivalence plot 202, the dotted line 201, and the dashed line 203. The first region 204 corresponds to high process outlet temperatures and relatively low L/G ratios, the second region 206 corresponds to lower process outlet temperatures and relatively higher L/G ratios, the third region 208 corresponds to relatively low process outlet temperatures and relatively low L/G ratios, and the fourth region 210 corresponds to relatively low process temperatures and relatively high L/G ratios. Although there is overlap between process outlet temperatures and/or L/G ratios included in the various regions, each region defines a different combination of process outlet temperatures and L/G ratios.

FIG. 2 further includes a key 212 that defines different spray-dried dispersions according to different symbols. Upright open triangle symbols represent L-grade HPMCAS formulations from the prior art. Open diamond symbols represent hydroxypropyl methylcellulose (HPMC) formulations from the prior art. Upright filled triangle symbols represent polyvinylpyrrolidone (PVP) formulations from the prior art. Downward filled triangles represent 100% active formulations from the prior art. Circles represent spray-dried HPMCAS-H from solvent systems comprising DMSO, according to the present disclosure, and V symbols represent spay-dried dispersions of vemurafenib, an active pharmaceutical ingredient, with HPMCAS-H from solvent systems comprising DMSO, according to the present disclosure. It may be understood that the dispersions are mapped according to the mass fraction of DMSO in the solvent feed.

As mentioned above, it is desired to perform the spray drying process with the process outlet temperature less than the wet T_{g} in order to avoid poor yields and physical instability of the spray-dried product. As such, the equivalence plot 202 may define an upper threshold for the process outlet temperature as a function of the L/G ratio and defines a desired processing space of the process map 200. Because the fourth region 210 is positioned entirely above the equivalence plot 202, spray drying using conditions (e.g., combinations of L/G ratio and Tₒᵤₜ) within the fourth region 210 is not desirable.

Further, performing the spray drying process using conditions within the first region 204 is not desirable due to the relatively low L/G ratios, which decreases throughput. In addition, the relatively high temperatures (e.g., 90° C. and higher) may result in physical instability and/or chemical degradation when the material being spray dried is temperature sensitive. When the material being spray dried is not temperature sensitive, spray drying using conditions in the second region 206 instead of the first region 204 may increase throughput due to the higher L/G ratios in the second region 206. Notably, the prior art formulations are concentrated within the first region 204. However, it may be understood that the dry T_{g} of the formulations shown may exceed that of pure HPMCAS-H in some examples. For example, some polymers contain PVP, which has a higher T_{g} than HPMCAS, while others contain an active pharmaceutical ingredient that may raise the T_{g} of the formulation (e.g., as evidenced by the fact that it can be spray dried with Tₒᵤₜ=120° C.).

As another example, performing the spray drying process using conditions within the third region 208 may enable temperature sensitive formulations, such as some biologics, to be spray-dried, although at a decreased throughput. However, it may be desirable to spray dry formulations having less temperature sensitivity using conditions within the second region 206, which includes both lower outlet temperatures (e.g., lower than the first region 204) and higher throughput (e.g., higher than the first region 204).

Notably, there is a maximum throughput (e.g., where the equivalence plot 202 is maximal) corresponding to an outlet temperature of 78° C. and an L/G ratio of 0.0128. Because the process conditions may vary during operation, using the equivalence plot 202 to define the process conditions also enables process optimization based on robustness considerations. For example, by reducing the L/G ratio 3% from the maximum of 0.0128 at 78° C. (e.g., to 0.0124 at 78° C.), fluctuations in the process outlet temperature of up to 5° C. may be tolerated. That is, the L/G ratio may be maximized so that fluctuations in Tₒᵤₜ at the maximized L/G ratio do not cause the wet T_{g} to exceed Tₒᵤₜ. Alternatively, the robustness may be considered for fluctuations in the L/G ratio at a constant value of Tₒᵤₜ. As an example, the process may be optimized for maintaining the L/G ratio at a constant value or for maintaining the Tₒᵤₜ at a constant value based on how precisely the L/G ratio or the Tₒᵤₜ may be controlled in a specific process.

In some examples, the second region 206 may be defined by a lower spray drying mass ratio (e.g., L/G ratio) threshold and an upper process outlet temperature threshold in addition to the equivalence plot 202. The lower spray drying mass ratio threshold and the upper process outlet temperature threshold may be single values or may vary. For example, the upper process outlet temperature threshold may be linear or may curve with respect to the L/G ratio, and/or vice versa (e.g., the lower mass ratio threshold may vary with respect to the process outlet temperature). The second region 206 may include L/G ratios that are greater than the lower spray drying mass ratio threshold and process outlet temperatures that are less than the upper process outlet temperature threshold. As a non-limiting example, the lower spray drying mass ratio threshold may be 0.004, such as shown by dashed line 203, which defines the border between the second region 206 and the third region 208 in the present example. The process conditions within the second region 206 may be favorable for spray drying small molecule-based pharmaceutical formulations, whereas the processing conditions within the third region 208 may be favorable for spray drying biologics, as mentioned above. As another non-limiting example, the upper process outlet temperature threshold may be a value within a range from 80-90° C. (e.g., 85° C.).

As another example, additionally or alternatively, the second region 206 and the third region 208 may be combined such that the second region 206 includes all of the process space outside of the first region 204 that is below the equivalence plot 202. In such an example, the lower mass ratio threshold may be determined based on the process outlet temperature relative to a pre-determined temperature threshold. The pre-determined temperature threshold may differentiate process conditions for small molecule-based formulations from those for biologic-based formulations. The lower mass ratio threshold may be higher when the process outlet temperature is greater than or equal to the pre-determined temperature threshold and lower when the process outlet temperature is less than the pre-determined temperature threshold. As an example, the pre-determined temperature threshold may be 60° C. In the present example, the lower spray drying mass ratio threshold may be 0.004 when the process outlet temperature is greater than or equal to 60° C., and the lower spray drying mass threshold mass ratio may be 0.001 when the outlet temperature is less than 60° C.

Although the process map 200 is shown for a system of HPMCAS-H, a similar methodology may be applied to a formulation containing an active pharmaceutical ingredient and a polymeric excipient. Depending on whether the T_{g} of the active pharmaceutical ingredient is higher or lower than that of the excipient and how the active pharmaceutical ingredient interacts with the solvent (e.g., determined via dynamic vapor sorption), the equivalence plot 202 may shift up or down, respectively. As an example, if the active pharmaceutical ingredient causes the formulation to retain more or less solvent at an equivalent relative saturation compared with the excipient alone, the equivalence plot 202 may shift down or up, respectively, relative to the excipient alone. Further, this methodology extends to mixed solvent systems, which combine a high volatility solvent with DMSO (and/or another low volatility solvent). In particular, the solubility of the active pharmaceutical ingredient (e.g., API) in the mixed solvent system may decrease more slowly with respect to decreasing the proportion of DMSO than the potential increase in the L/G ratio (e.g., the spray drying throughput) that may be enabled by decreasing the amount of DMSO in the mixture. This strategy may seek to maximize process throughput, which is defined as the quantity obtained by multiplying the active pharmaceutical ingredient concentration and the liquid feed rate (e.g., the amount of API processed per unit time). For example, if when compared to a 100% DMSO solvent system, solubility of the active pharmaceutical ingredient decreases by 20% in 50/50 DMSO/acetone but the L/G ratio doubles, then the API processing rate will increase by using the mixed solvent system.

Alternatively, the active pharmaceutical ingredient concentration may be limited by a maximum allowable excipient concentration for spray drying (e.g., due to viscosity limitations and ability to atomize). In such examples, throughput may be maximized by setting the DMSO composition of the solvent system to the minimum level that enables the maximum acceptable excipient concentration. For example, it may be that the maximum desirable active pharmaceutical ingredient concentration is 2% by weight because of solution viscosity considerations. In the present example, if a solvent system of 50/50 DMSO/acetone enables spray drying at 2% by weight of the active pharmaceutical ingredient, then there would be no advantage to using higher DMSO ratios even if 100% DMSO could dissolve 20% by weight of the active pharmaceutical ingredient, for example.

Similar to the approach demonstrated for pure DMSO, the wet T_{g} may be determined as a function of process outlet temperature for any mixture of active pharmaceutical ingredient, excipient, and one or more solvents. In practice, however, because the high volatility solvent is typically so much more volatile than DMSO, the shape of the equivalence plot 202 is dominated by the thermodynamic properties of DMSO. For example, the “optimal” process outlet temperature of 78° C. implied by the equivalence plot 202 in FIG. 2 exceeds the boiling points of common spray drying solvents like acetone, methanol, DCM, and THF. Thus, the methodology described herein may be used to define acceptable process outlet temperature ranges for spray drying out of solvents containing DMSO as a function of throughput (e.g., the L/G ratio), resulting in significant increases in throughput combined with significant decreases in the process outlet temperature. An example of determining the wet T_{g} will be described below with reference to FIGS. 5-6.

Although the process map 200 provides information with respect to throughput, it does not provide information regarding yield for each spray-drying process. Such information is provided in FIGS. 3 and 4. Turning first to FIG. 3, an example yield plot 300 depicting percent yield (vertical axis) as a function of predicted values of T_{g}−Tₒᵤₜ (e.g., a difference between the predicted T_{g} and Tₒᵤₜ, as shown on the horizontal axis) for 10 g batches of HPMCAS-H sprayed out of DMSO solvent blends is shown. The data of the yield plot 300 is separated by the high-volatility co-solvent used in the solvent blend according to a key 302. For example, data points indicated by open circle symbols are for HPMCAS-H samples sprayed out of a solvent blend comprising DMSO and THF, and data points indicated by open triangle symbols are for HPMCAS-H samples sprayed out of a solvent blend comprising DMSO and acetone. The yield plot 300 further includes an equivalence line 304 where T_{g} is equal to Tₒᵤₜ, which may correspond to the equivalence plot 202 of FIG. 2.

Specifically, the percent yield is mapped according to the difference between T_{g} and Tₒᵤₜ used in the spray drying process. The yield plot 300 indicates that for positive values of T_{g}−Tₒᵤₜ (e.g., where the process outlet temperature used during the spray drying is kept below the predicted wet glass transition temperature of the spray-dried material), the yield is relatively insensitive to the magnitude of T_{g}−Tₒᵤₜ. The yield fluctuates around a mean value of 79.7%, with a standard deviation of 8.6% for these positive values of T_{g}−Tₒᵤₜ, although it may be understood that yields may vary depending on the specific spray dryer used, the batch size, and the method of atomization used. Thus, it may be understood that the yield remains relatively unchanged while spray drying with processing conditions where Tₒᵤₜ does not exceed T_{g}.

Furthermore, the yield plot 300 of FIG. 3 indicates that yield begins to decrease as the value of T_{g}−Tₒᵤₜ becomes increasingly negative. That is, the yield decreases as the process outlet temperature of the spray dryer further increases above the predicted wet glass transition temperature of the spray-dried material, as expected. For example, at a value of (T_{g}−Tₒᵤₜ)=−16° C., the yield for one spray was approximately 0% while the yield for another was at 45%, which is well below the average yield when T_{g}−Tₒᵤₜ is positive. Thus, the yield plot 300 shows that yield is relatively unaffected by T_{g}−Tₒᵤₜ when T_{g}−Tₒᵤₜ is positive, enabling the use of lower process outlet temperatures relative to the “optimal” temperature where throughput is maximized (see FIG. 2) without decreasing the yield.

This conclusion is further supported by an additional example yield plot 400 shown in FIG. 4. Similarly, the yield plot 400 depicts percent yield (vertical axis) as a function of predicted values of T_{g}−Tₒᵤₜ for 5 g batches of HPMCAS-H sprayed out of 100% DMSO or DMSO solvent blends, as indicated by a key 402. For example, data points indicated by open circle symbols are for HPMCAS-H samples sprayed out of a solvent blend comprising DMSO and THF, data points indicated by open triangle symbols are for HPMCAS-H samples sprayed out of a solvent blend comprising DMSO and acetone, and data points indicated by open diamond symbols are for HPMCAS-H samples sprayed out of 100% DMSO. The yield plot 400 further includes an equivalence line 404 where T_{g} is equal to Tₒᵤₜ, which may correspond to the equivalence plot 202 of FIG. 2.

Similar to the yield plot 300 of FIG. 3, the yield plot 400 of FIG. 4 demonstrates decreasing yields as negative values of T_{g}−Tₒᵤₜ increase. Thus, although the magnitudes of the yields between the yield plot 300 of FIG. 3 and the yield plot 400 of FIG. 4 are not directly comparable due to the difference in batch size, the yield plot 400 supports the methodology described herein for increasing both yield and throughput by operating with the process outlet temperatures below the glass transition temperature, as determined as a function of the L/G ratio.

In order to determine the relationship between the L/G ratio and the equivalence plot, such as shown in FIG. 2, T_{g} is determined as a function of relative saturation of DMSO. The relative saturation of DMSO refers to an amount of DMSO in the vapor phase relative to a total amount of DMSO that can be held in a drying gas stream, where 100% refers to fully saturated drying gas. For example, up to approximately 50% relative saturation of DMSO may be targeted. Referring now to FIG. 5, an example dynamic vapor sorption (DVS) plot 500 is shown, which may be used to define an amount of DMSO uptake in a material as a function of DMSO relative saturation. In particular, the DVS plot 500 shows a percentage weight increase of a sample relative to an initial mass of the sample (vertical axis) as a function of the DMSO relative saturation (horizontal axis). Increases in the weight of the sample may be attributed to DMSO uptake by the sample. In the present example, the sample comprises HPMCAS-H; however, it may be understood that other materials may be examined similarly, such as different excipients, active pharmaceutical ingredients, and their combinations.

The DVS plot 500 includes a lower curve 502 corresponding to DMSO adsorption onto HPMCAS-H and an upper curve 504 corresponding to DMSO desorption by the HPMCAS-H at a constant temperature (e.g., 50° C.). As the relative saturation of DMSO increases, the amount of DMSO uptake by the HPMCAS-H increases by a measurable amount. Each curve connects four data points corresponding to different DMSO relative saturations, although a different number of data points and and/or samples at different DMSO relative saturations may be used. Samples of the DMSO-laden HPMCAS-H at a plurality of DMSO relative saturations may be removed from an instrument performing the DVS analysis (e.g., a gravimetric instrument) and further analyzed to determine their glass transition temperatures (e.g., the wet T_{g}), such as via differential scanning calorimetry (e.g., modulated differential scanning calorimetry). For example, samples at each examined DMSO relative saturation may be analyzed.

Turning now to FIG. 6, a plot 600 shows T_{g} (vertical axis) as a function of DMSO relative saturation (horizontal axis) for HPMCAS-H. As noted above with respect to FIG. 5, although HPMCAS-H is used in the present example, other materials may be examined similarly to determine the relationship between T_{g} and the DMSO relative saturation. The plot 600 includes a curve 602 connecting data points for each DMSO relative saturation, which correspond to the DMSO relative saturation samples from the DVS plot 500 of FIG. 5.

The plot 600 of FIG. 6 may be then used to determine the relationship between the L/G ratio and the difference between T_{g} and Tₒᵤₜ by noting that a mass and energy balance performed on the spray dryer, combined with the known vapor-liquid equilibrium behavior of DMSO, can be used to establish a relationship between the L/G ratio and the DMSO relative saturation at a given value of Tₒᵤₜ. Therefore, at a given L/G ratio and Tₒᵤₜ, the relative saturation of DMSO and thus the predicted wet T_{g} may be calculated. An equivalence plot, such as the equivalence plot 202 of FIG. 2, can therefore be determined by holding the L/G ratio fixed and varying Tₒᵤₜ until it matches the predicted wet T_{g}.

It may be understood that the calculation outlined above can be performed either by numerically interpolating between the experimental data points and/or by fitting the data to models to use analytical optimization techniques. A practical advantage of the analytical approach is that it is less sensitive to experimental noise.

Next, FIG. 7 shows an example method 700 for identifying processing conditions for preparing a pharmaceutical formulation comprising an active pharmaceutical ingredient (e.g., a drug) and excipient(s) via spray drying with at least one low volatility solvent. Because the spray drying process is used to remove solvent and low volatility solvents are inherently more difficult to evaporate than high volatility solvents, the method 700 may enable optimization of the low volatility solvent usage for each pharmaceutical formulation based on thermodynamic considerations that increase drug solubility and stability while decreasing residual solvent in the spray-dried formulation and kinetic considerations that make the preparation economically viable by increasing throughput and yield.

At 702, the method 700 includes determining a first relationship between a relative saturation of the low volatility solvent (e.g., DMSO) and a glass transition temperature of the material to be spray dried. In some examples, the first relationship may be determined for a combination of the active pharmaceutical ingredient and the excipient(s), such as according to their ratio in the pharmaceutical formulation or another ratio that enabled the first relationship to be empirically determined. In other examples, the first relationship may be determined for the excipient(s) alone or the active pharmaceutical ingredient alone, such as whether the glass transition temperature of the active pharmaceutical ingredient is higher or lower than that of the excipient.

Further, in some examples, the relationship between the relative saturation and the glass transition temperature may be determined for mixed solvent systems. For example, the mixed solvent system may include more than one low volatility solvent mixed in a defined ratio or the low volatility solvent(s) mixed with one or more high volatility solvent(s) in a defined ratio. As such, although the method 700 will be described for a low volatility solvent, it may be understood that the method 700 also may be applied to mixed solvent systems.

Determining the first relationship may include defining an amount of uptake of the low volatility solvent(s) by the material as a function of the relative saturation of the low volatility solvent(s), such as via dynamic vapor sorption. An example of a dynamic vapor sorption plot is described above with respect to FIG. 5. Determining the first relationship may further include analyzing the material at different relative saturations of the low volatility solvent(s) via modulated differential scanning calorimetry to determine a corresponding glass transition temperature, such as described above with respect to FIG. 6.

Examples of different excipients that may be used include any combination of HPMCAS, HPMC, PVP, polyvinylpyrrolidone-vinyl acetate copolymer (PVP-VA), and various polymethacrylates (e.g., Eudragit® L100 or L100-55) with any combination of DMSO, NMP, DMF, and DMAC. In some examples, DMSO may be a desired low volatility solvent due to its low toxicity and relatively high allowable concentration in a final pharmaceutical product. DMF and/or DMAC, while still being low volatility solvents, have a vapor pressure as much as 5 times higher than DMSO in a common spray drying temperature range of 50° C. to 100° C. For this reason, in alternate examples, DMF and/or DMAC may be desired over DMSO for improving a spray drying yield and/or throughput. Further, in some examples, up to a threshold percentage (e.g., 5%) of water or methanol may be included to increase polymer solubility in some solvent systems.

At 704, the method 700 includes determining a second relationship between a spray drying mass ratio (e.g., a liquid mass to gas mass, or L/G, ratio) and the relative saturation of the low volatility solvent at a given process outlet temperature based on the first relationship. For example, the known vapor-liquid equilibrium behavior of the low volatility solvent may be used to establish a relationship between the L/G ratio and the low volatility solvent relative saturation at a given process outlet temperature, such as via mass and energy balance relationships.

At 706, the method 700 includes calculating a predicted glass transition temperature for the material to be spray dried at a given spray drying mass ratio based on the second relationship. For example, the amount of the low volatility solvent adsorbed by the material affects the glass transition temperature, and thus, the relative saturation of the low volatility solvent at the given spray drying mass ratio may be determined.

At 708, the method 700 includes generating a process map relating the process outlet temperature to the spray drying mass ratio based on the predicted glass transition temperature at each spray drying mass ratio. This includes generating an equivalence plot where the predicted glass transition temperature equals the process outlet temperature at each spray drying mass ratio, as indicated at 710. The equivalence plot may be generated by interpolating between data points obtained while determining the first relationship and the second relationship or via analytical optimization techniques by fitting the data points to models, such as mentioned above with respect to FIG. 6.

At 712, the method 700 includes identifying a process outlet temperature corresponding at maximum throughput (e.g., maximum spray drying mass ratio) based on the equivalence plot. The process outlet temperature at maximum throughput may be identified as a global maximum of the equivalence plot, where the equivalence plot peaks with respect to the L/G ratio.

At 714, the method 700 includes performing the spray drying with parameters (e.g., processing parameters) optimized based on the process outlet temperature at the maximum throughput as well as solubility and viscosity constraints, as will be elaborated below with respect to FIG. 8. For example, the maximum L/G ratio may be used, and the process outlet temperature used may be no greater than the glass transition temperature at the maximum L/G ratio. As another example, a different L/G ratio may be used that is optimized for the material to be spray dried according to solubility and/or viscosity constraints, if present, and the process outlet temperature may be no greater than the glass transition temperature at the optimized L/G ratio. The method 700 then ends. For example, the method 700 may be repeated for each pharmaceutical formulation in order to optimize the processing throughput of that formulation.

Referring now to FIG. 8, an example method 800 is shown for using a low volatility solvent as a processing solvent for preparing a pharmaceutical formulation comprising a drug (e.g., an active pharmaceutical ingredient) and excipient(s) via spray drying. Because the spray drying process is used to remove solvent and low volatility solvents are inherently more difficult to evaporate than high volatility solvents, the method 800 may utilize optimized conditions for the spray drying of each pharmaceutical formulation based on thermodynamic considerations that increase drug solubility and stability while decreasing residual solvent in the spray-dried formulation and kinetic considerations that make the preparation economically viable by increasing throughput and yield. For example, the method 800 may utilize process conditions determined via the method 700 of FIG. 7.

At 802, the method 800 includes selecting one or more low volatility solvents, one or more high volatility solvent(s), and a solvent ratio of the low volatility solvent(s) and high volatility solvent(s) based on a spray drying mass ratio and process outlet temperature at maximum throughput (see FIG. 7), a solubility of the material to be spray dried, and a viscosity of the material to be spray dried. As described above, the low volatility solvent(s) may be selected from DMSO, NMP, DMF, and DMAC, for example, while the high volatility solvent(s) may be selected from acetone, methanol, ethanol, isopropanol, ACN, THF, DCM, EtOAc, and water. DMSO may be preferentially selected as the low volatility solvent due to the high residual solvent threshold for DMSO relative to the other low volatility solvents, which may reduce a burden for aggressive secondary drying that may be used with other solvents that have lower residual solvent threshold. Further, DMSO readily dissolves both polar and non-polar compounds and is miscible with water and many organic solvents, such as the high volatility solvents described herein, giving it broad utility.

The amount of the low volatility solvent(s) in the solvent system may range from 0-100%. For example, the solvent system may include a first percentage of the low volatility solvent(s) and a second, remaining percentage of the high volatility solvent(s). As an example, the low volatility solvent(s) may comprise at least 10% of the solvent system (e.g., by weight or by volume). However, as the percentage of the low volatility solvent(s) in the solvent system increases, a process outlet temperature for the spray drying may increase and/or a process rate (e.g., a liquid feed rate) of the spray drying may decrease in order to aid the solvent removal.

Therefore, for the process to be viable in terms of yield and throughput, it is desired for the resulting solution of the drug and excipient(s) in the solvent system to be processed at an approximately maximum L/G ratio and with a process outlet temperature that is selected as a function of the L/G ratio. As mentioned above, the L/G ratio refers to a ratio of the liquid mass feed rate (L) to a drying gas mass feed rate (G) supplied to the spray dryer, such as described with respect to FIGS. 1-6. Further, the process outlet temperature may be set to reduce chemical and physical degradation during the spray drying and may be less than the boiling point of the low volatility solvent(s). As such, the L/G ratio and the process outlet temperature may be selected based on processing constraints related to the wet T_{g} of the material being spray dried.

Through empirical testing, it may be determined that using above a threshold percentage of a particular low volatility solvent may decrease the L/G and/or demand the process outlet temperatures to be greater than the wet T_{g} of the material being spray dried in order to remove the low volatility solvent to acceptable levels (e.g., where the dispersion is solid, physically stable so that it is not prone to recrystallization during process-relevant time scales, and any remaining solvent is at or below a desired residual solvent threshold level). Further, as mentioned above, the residual solvent threshold level may be different for different low volatility solvents, such as higher for DMSO. Therefore, percentages of the particular low volatility solvent that are above the residual solvent threshold level for that solvent may not be considered.

The solubility and viscosity of the material to be spray dried are also considered in selecting the low volatility solvent(s), the high volatility solvent(s), and their ratio in order to maximize process throughput. In particular, decreasing the proportion of the low volatility solvent(s) in the solvent system may decrease the solubility of the active pharmaceutical ingredient but increase the L/G ratio. As one example, the solubility may decrease more slowly than the L/G ratio increases as the proportion of the low volatility solvent(s) in the solvent system decreases. In such examples, a concession in solubility may be made in favor of the L/G ratio in order to increase process throughput.

In still other examples, the active pharmaceutical ingredient concentration may be limited by a maximum allowable excipient concentration in the solvent system for spray drying due to viscosity limitations. In such examples, the spray drying mass ratio may be maximized by setting the proportion of the low volatility solvents(s) in the solvent system to the minimum level that enables the highest drug concentration. For example, there may be no advantage to using higher proportions of the low volatility solvent(s) beyond that enabling the maximum allowable excipient concentration in the solvent system even if increasing the portion of the low volatility solvent(s) would further increase the solubility of the materials to be spray dried.

As such, a plurality of solvent blends of varying ratios of low volatility solvent(s) and high volatility solvent(s) may be considered. When there is comparable solubility in multiple candidate solvent blends, DMSO may be preferentially selected as the low volatility solvent, as discussed above. For example, a first solvent system that includes DMSO as the low volatility solvent may be selected when the solubility of the drug is similar in the first solvent system compared with a second solvent system with a different low volatility solvent (e.g., not including DMSO). Further, when multiple percentages of the low volatility solvent(s) produce comparable solubility, the lowest percentage may be selected, as the high volatility solvent(s) are more readily removable via spray drying. As used herein, the term “comparable” may denote solubility values that are substantially the same. As another example, the term “comparable” may denote solubility values that are within a threshold percentage of each other, such as within 1-20%.

At 804, the method 800 includes selecting the process outlet temperature and spray drying mass ratio based on the predicted glass transition temperature of the material to be spray dried as a function of the processing mass ratio and a temperature sensitivity of the material to be spray dried. For example, the selected process outlet temperature may not be greater than the predicted glass transition temperature of the material to be spray dried at a given spray drying mass ratio. In some examples, the selected process outlet temperature may be decreased from the predicted glass transition temperature in order to tolerate fluctuations in the process outlet temperature that may occur during the spray drying and/or due to the temperature sensitivity of the material to be spray dried. In such examples, the selected process outlet temperature may be centered within the predicted fluctuations so that any temperature fluctuations during the spray drying may not increase the process outlet temperature above the predicted glass transition temperature or a degradation temperature of the material.

At 806, the method 800 includes preparing the solvent system according to the selected low volatility solvent(s), the selected high volatility solvent(s), and the selected ratio of the low volatility solvent(s) and high volatility solvent(s) (e.g., as determined at 802). The solvent system may be prepared on a relatively large scale. For example, it may be desired to process at least 1 kilograms (kg) of the drug, and so enough of the solvent system may be prepared to bring 1 kg of the drug into solution. For example, the amount of the solvent system to prepare may be estimated based on the solubility studies described above at 802.

At 808, the method 800 includes adding the drug and the excipient(s) to the solvent system to form a solution. The solution may be agitated to aid in mixing. In some examples, the method 800 includes heating the solution to increase the solubility, as optionally indicated at 810. For example, a temperature of the heating may be kept below the boiling point of the solvent system as well as below a degradation temperature of the drugs and excipient(s).

At 812, the method 800 includes spray drying the solution at the selected spray drying mass ratio and the selected process outlet temperature to form a dry amorphous dispersion. As mentioned above with respect to FIG. 1, the solution is pumped through an atomizing nozzle of the spray dryer at a liquid feed rate of the selected spray drying mass ratio, and the atomizing nozzle converts the solution to small-sized (e.g., microns to hundreds of microns) droplets that come into contact with a heated drying gas that is fed to the spray dryer at a gas feed rate of the selected spray drying mass ratio.

During the spray drying, the method 800 includes operating the spray dryer with the process outlet temperature below the predicted glass transition temperature (e.g., upper threshold process outlet temperature) of the material, as indicated at 814. The upper threshold process outlet temperature may be based on chemical and physical stability constraints of the material. As defined above at 804, operating below the predicted glass transition temperature may decrease physical instability (e.g., crystallization and/or phase separation) of the spray dried product. Further, in some systems, operating below the predicted glass transition temperature may also decrease a chemical degradation of the spray dried product.

The method 800 may then end. For example, the method 800 may be repeated for each pharmaceutical formulation in order to spray dry the pharmaceutical formulation using optimized process parameters. Further, the spray-dried amorphous dispersion may be further dried via a secondary drying technique, such as tray drying or vacuum drying.

Methods 700 and 800 may be applied to spray drying a plurality of combinations of low volatility solvent, high volatility solvent, drug, and excipient. Examples of possible combinations along with resulting yields are shown in table 1 below. The spray dried products collected for each of the examples given in table 1 were characterized as amorphous free-flowing

powders and no significant buildup on the spray dryer was observed.

TABLE 1
Spray drying parameters and outcomes when following method 700 and 800. VF is
vemurafenib, SL is spironolactone, FS is furosemide, HT is hydrochlorothiazide.

| | | wt. % drug | Batch | LV | HV | | solids | | |
|---|---|---|---|---|---|---|---|---|---|
| | | in | Size | solvent | solvent | Yield | loading | | Tₒᵤₜ |
| Drug | Excipient | dispersion | (g) | (wt. %) | (wt. %) | (%) | (wt. %) | L/G | (° C.) |
| VF | HPMCAS-H | 14 | 7 | DMSO | THF | 64 | 7 | 0.0064 | 70 |
| | | | | (25%) | (75%) | | | | |
| VF | HPMCAS-H | 14 | 7 | DMSO | Acetone | 51 | 7 | 0.0127 | 70 |
| | | | | (50%) | (50%) | | | | |
| SL | HPMCAS-H | 20 | 20 | DMSO | Acetone | 55 | 10 | 0.0075 | 75 |
| | | | | (25%) | (75%) | | | | |
| FS | HPMCAS-H | 20 | 20 | DMSO | Acetone | 50 | 10 | 0.0077 | 75 |
| | | | | (25%) | (75%) | | | | |
| HT | HPMCAS-H | 20 | 20 | DMSO | Acetone | 55 | 10 | 0.0075 | 75 |
| | | | | (25%) | (75%) | | | | |
| dBET1 | n/a | 100 | 1 | DMSO | Acetone | 42 | 15 | 0.0071 | 75 |
| | | | | (25%) | (75%) | | | | |
| HT | HPMCAS-H | 20 | 20 | DMF | Acetone | 78 | 10 | 0.0075 | 75 |
| | | | | (25%) | (75%) | | | | |
| HT | PVP-VA | 20 | 20 | DMAc | Acetone | 80 | 10 | 0.0075 | 75 |
| | | | | (25%) | (75%) | | | | |
| HT | Eudragit ® | 20 | 20 | DMSO | Acetone | 92 | 6 | 0.0063 | 70 |
| | L100 | | | (25%) | (75%) | | | | |

As shown in table 1, a plurality of drugs, excipients and low volatility solids may be used following method 700 and 800 to produce a spray dried dispersion with a yield of at least 50%. In an example where dBET1 is the drug, enhancing solubility by including up to 25% DMSO allows for spray drying of a 100% drug dispersion. In such an example, a yield of 42% was achieved with a relatively small batch size of 1 g. Additionally, two examples are shown in table 1 where hydrochlorothiazide is spray dried with DMSO as the low volatility solvent and where spray drying parameters are kept constant and DMSO is replaced with DMF. Comparing the two examples shows that replacing DMSO with the more volatile (e.g., higher vapor pressure) DMF results in an increase in yield from 55% to 78%.

Conversely, selecting spray drying mass ratios and outlet process temperatures outside the maximum spray drying mass ratios and outlet temperatures as shown in methods 700 and 800 may result in a sticky, wet, and non-free flowing product and a decrease in product yield as shown below in table 2.

TABLE 2
Spray drying parameters and outcomes when not following
method 700 and 800. VF is vemurafenib.

| | | wt. % drug | Batch | LV | HV | | solids | | |
|---|---|---|---|---|---|---|---|---|---|
| | | in | Size | solvent | solvent | Yield | loading | | Tₒᵤₜ |
| Drug | Excipient | dispersion | (g) | (wt. %) | (wt. %) | (%) | (wt. %) | L/G | (° C.) |
| VF | HPMCAS-H | 15 | 10 | DMSO | THF | 35 | 10 | 0.0150 | 70 |
| | | | | (25%) | (75%) | | | | |

As an example, shown in table 2, the L/G of 0.0150 chosen for spray drying the formulation of vemurafenib is greater than maximum spray drying mass ratio. The resulting product from spray drying and the L/G ratio above the maximum spray drying ratio may be wet, sticky, and not free flowing. Such product may stick inside a spray dryer and decrease a product yield.

Referring now to FIG. 9, an example process map 900 shows conditions for spray drying a material with respect to favorability in terms of increasing both throughput and yield. The intensity (e.g., darkness) of the shading increases as the process conditions become less favorable or desirable, such as due to decreasing throughput and/or yield. The vertical axis of the process map 900 represents the L/G ratio, and the horizontal axis represents the outlet temperature.

Even if the throughput is high due to a high L/G ratio, the yield may be decreased by spray drying with an outlet temperature that is greater than the glass transition temperature of the material at the given L/G ratio. For example, an equivalence plot where the outlet temperature is equal to the glass transition temperature may define at least a portion of an upper boundary for a lightest (e.g., least shaded) region 902 having the highest favorability in terms of both throughput and yield. Further, the favorability may decrease as the L/G ratio increases above the equivalence plot, as demonstrated by the increased shading at higher L/G ratios. As another example, the favorability may generally decrease, and thus the shading becomes more intense, as the outlet temperature increases due to chemical and/or physical instability of the material at higher outlet temperatures. Although the yield may be unaffected, the favorability generally decreases as the L/G ratio decreases due to the decreased throughput, as shown by the increased shading intensity at lower L/G ratios.

In this way a low volatility solvent may be combined with a high volatility solvent in order to achieve large increases in drug solubility that enable processes to become economically viable at a commercial scale. The technical effect of increasing a solubility of a drug in a solution processed through spray drying by using a mixture of a low volatility solvent and a high volatility solvent at an optimized ratio and process temperature is that a yield and quality (e.g., solvent levels below a threshold and not physically or chemically degraded) of a resulting spray-dried dispersion of the drug may be increased.

The disclosure provides support for a method for preparing a dispersion of a pharmaceutical formulation, comprising: selecting a solvent mixture comprising a first percentage of a low volatility solvent and a second percentage of a high volatility solvent based on a solubility of a drug of the pharmaceutical formulation in the solvent mixture, forming a solution of the drug of the pharmaceutical formulation in the solvent mixture, and spray drying the solution to form the dispersion using a spray drying mass ratio that is selected based on a relationship between a glass transition temperature of the pharmaceutical formulation and a relative saturation of the low volatility solvent during the spray drying, the spray drying mass ratio defining a ratio of a liquid feed rate of the solution to a gas feed rate of a drying gas during the spray drying, and using a process outlet temperature that is less than the glass transition temperature of the pharmaceutical formulation. In a first example of the method, the spray drying mass ratio is further selected to maximize a processing throughput of the pharmaceutical formulation while spray drying the solution with the process outlet temperature that is less than the glass transition temperature, and wherein the glass transition temperature varies as a function of the spray drying mass ratio. In a second example of the method, optionally including the first example, the first percentage is at least 10%, and wherein selecting the solvent mixture is further based on kinetic and thermodynamic properties of removing the solvent mixture via spray drying. In a third example of the method, optionally including one or both of the first and second examples, the pharmaceutical formulation further comprises an excipient, and wherein the excipient is included in the solution in the solvent mixture and comprises at least one of hypromellose acetate succinate, hydroxypropyl methylcellulose, polyvinylpyrrolidone, polyvinylpyrrolidone-vinyl acetate copolymer, and polymethacrylate and the solvent mixture comprises at least one of dimethylsulfoxide (DMSO), n-methyl-2-pyrrolidone (NMP), dimethylformamide (DMF), and n,n-dimethylacetamide (DMAC). In a fourth example of the method, optionally including one or more or each of the first through third examples, dimethylsulfoxide (DMSO) is selected as the low volatility solvent based on the solubility of the drug being comparable in solvent blends including DMSO compared with solvent blends not including DMSO, and wherein the spray drying mass ratio is greater than a lower threshold spray drying mass ratio that is determined based on the process outlet temperature. In a fifth example of the method, optionally including one or more or each of the first through fourth examples, the lower threshold spray drying mass ratio is 0.004 with respect to DMSO when the process outlet temperature is greater than or equal to 60° C., and wherein the lower threshold spray drying mass ratio is 0.001 with respect to DMSO when the process outlet temperature is less than 60° C. In a sixth example of the method, optionally including one or more or each of the first through fifth examples, the process outlet temperature is less than an upper threshold process outlet temperature that is determined based on chemical and physical stability constraints of the pharmaceutical formulation. In a seventh example of the method, optionally including one or more or each of the first through sixth examples, the upper threshold process outlet temperature is 90° C.

The disclosure also provides support for a method for identifying processing parameters for spray drying a pharmaceutical formulation from a low volatility solvent, comprising: generating a process map relating a process outlet temperature of the spray drying to a spray drying mass ratio based on a glass transition temperature of the pharmaceutical formulation at each spray drying mass ratio, and generating an equivalence plot on the process map where the glass transition temperature of the pharmaceutical formulation equals the process outlet temperature as a function of the spray drying mass ratio. In a first example of the method, generating the process map relating the process outlet temperature of the spray drying to the spray drying mass ratio based on the glass transition temperature of the pharmaceutical formulation at each spray drying mass ratio comprises: determining a first relationship between a relative saturation of the low volatility solvent and the glass transition temperature of the pharmaceutical formulation, determining a second relationship between the spray drying mass ratio and the relative saturation of the low volatility solvent at a given process outlet temperature of the spray drying based on the first relationship, and determine the glass transition temperature of the pharmaceutical formulation at each spray drying mass ratio based on the second relationship. In a second example of the method, optionally including the first example, the method further comprises: identifying a maximum of the equivalence plot corresponding to a maximum spray drying mass ratio of the equivalence plot, and identifying a corresponding process outlet temperature at the maximum of the equivalence plot. In a third example of the method, optionally including one or both of the first and second examples, the method further comprises: selecting the maximum spray drying mass ratio and a process outlet temperature that is less than or equal to the corresponding process outlet temperature at the maximum of the equivalence plot as the processing parameters for spray drying the pharmaceutical formulation. In a fourth example of the method, optionally including one or more or each of the first through third examples, the method further comprises: selecting a portion of the low volatility solvent that is lower than a highest portion of low volatility solvent based on at least one of a solubility of the pharmaceutical formulation in the low volatility solvent, a viscosity of the pharmaceutical formulation in the low volatility solvent, and a temperature sensitivity of the pharmaceutical formulation. In a fifth example of the method, optionally including one or more or each of the first through fourth examples, the equivalence plot defines a maximum spray drying mass ratio as a function of the process outlet temperature for the spray drying.

The disclosure also provides support for a system for preparing a pharmaceutical formulation, comprising: a solution of a drug and an excipient dissolved in a solvent blend comprising at least one low volatility solvent and at least one high volatility solvent, wherein the at least one low volatility solvent comprises at least 10% of the solvent blend, and a spray dryer configured to receive the solution and a stream of drying gas and output a dried dispersion of the drug and the excipient as the pharmaceutical formulation, wherein a process outlet temperature of the spray dryer is selected as a function of a spray drying mass ratio of a liquid feed rate of the solution to a gas feed rate of the drying gas. In a first example of the system, the at least one low volatility solvent is selected from dimethylsulfoxide (DMSO), n-methyl-2-pyrrolidone (NMP), dimethylformamide (DMF), and n,n-dimethylacetamide (DMAC) and wherein at least one high volatility solvent is selected from acetone, methanol, ethanol, isopropanol, ethyl acetate, acetonitrile, tetrahydrofuran, methylene chloride, n-methyl tetrahydrofuran, n-propanol, chloroform, hexanes, acetic acid, cyclohexane, ethyl ether, and water. In a second example of the system, optionally including the first example, the process outlet temperature of the spray dryer is less than a glass transition temperature of the dried dispersion of the drug and the excipient. In a third example of the system, optionally including one or both of the first and second examples, a relative saturation of the at least one low volatility solvent at an outlet of the spray dryer at equilibrium is between 5 and 50%. In a fourth example of the system, optionally including one or more or each of the first through third examples, the spray drying mass ratio of the liquid feed rate of the solution to the gas feed rate of the drying gas is selected to maximize an amount of the drug and the excipient processed per unit of time. In a fifth example of the system, optionally including one or more or each of the first through fourth examples, the solvent blend is selected according to a solubility of the drug and the excipient in the solvent blend and a maximum spray drying mass ratio of the liquid feed rate of the solution to the drying gas using the solvent blend.

In an alternate embodiment, the disclosure also provides support for a pharmaceutical formulation spray dried by a process of: forming a solution of an active pharmaceutical ingredient and an excipient of the pharmaceutical formulation in a solvent system comprising a low volatility solvent, the solvent system selected based on a solubility of the active pharmaceutical ingredient in the low volatility solvent and kinetic and thermodynamic properties of removing the low volatility solvent during spray drying, and spray drying the solution using a spray drying mass ratio that is selected based on a relationship between a glass transition temperature of the pharmaceutical formulation and a relative saturation of the low volatility solvent during the spray drying, the spray drying mass ratio defining a liquid feed rate of the solution to a gas feed rate of a drying gas during the spray drying, and a process outlet temperature that is less than the glass transition temperature of the pharmaceutical formulation. In a first example of the system, the spray drying mass ratio used during spray drying the pharmaceutical formulation is further selected to maximize a processing throughput of the pharmaceutical formulation while spray drying the solution with the process outlet temperature that is less than the glass transition temperature, and wherein the glass transition temperature varies as a function of the spray drying mass ratio. In a second example of the system, optionally including the first example, the low volatility solvent comprises at least 10% of the solvent system. In a third example of the system, optionally including one or both of the first and second examples, the low volatility solvent comprises dimethylsulfoxide (DMSO). In a fourth example of the system, optionally including one or more or each of the first through third examples, the excipient comprises at least one of hypromellose acetate succinate, hydroxypropyl methylcellulose, polyvinylpyrrolidone, polyvinylpyrrolidone-vinyl acetate copolymer, and polymethacrylate. In a fifth example of the system, optionally including one or more or each of the first through fourth examples, the low volatility solvent further comprises at least one of n-methyl-2-pyrrolidone (NMP), dimethylformamide (DMF), and n,n-dimethylacetamide (DMAC) and wherein the solvent system further comprises at least one of acetone, methanol, ethanol, isopropanol, ethyl acetate, acetonitrile, tetrahydrofuran, methylene chloride, n-methyl tetrahydrofuran, n-propanol, chloroform, hexanes, acetic acid, cyclohexane, ethyl ether, and water.

The following claims particularly point out certain combinations and sub-combinations regarded as novel and non-obvious. These claims may refer to “an” element or “a first” element or the equivalent thereof. Such claims should be understood to include incorporation of one or more such elements, neither requiring nor excluding two or more such elements. Other combinations and sub-combinations of the disclosed features, functions, elements, and/or properties may be claimed through amendment of the present claims or through presentation of new claims in this or a related application. Such claims, whether broader, narrower, equal, or different in scope to the original claims, also are regarded as included within the subject matter of the present disclosure.

1. A method for preparing a dispersion of a pharmaceutical formulation, comprising:
selecting a solvent mixture comprising a first percentage of a low volatility solvent and a second percentage of a high volatility solvent based on a solubility of a drug of the pharmaceutical formulation in the solvent mixture;
forming a solution of the drug of the pharmaceutical formulation in the solvent mixture; and
spray drying the solution to form the dispersion using a spray drying mass ratio that is selected based on a relationship between a glass transition temperature of the pharmaceutical formulation and a relative saturation of the low volatility solvent during the spray drying, the spray drying mass ratio defining a ratio of a liquid feed rate of the solution to a gas feed rate of a drying gas during the spray drying, and using a process outlet temperature that is less than the glass transition temperature of the pharmaceutical formulation.

2. The method of claim 1, wherein the spray drying mass ratio is further selected to maximize a processing throughput of the pharmaceutical formulation while spray drying the solution with the process outlet temperature that is less than the glass transition temperature, and wherein the glass transition temperature varies as a function of the spray drying mass ratio.

3. The method of claim 1, wherein the first percentage is at least 10%, and wherein selecting the solvent mixture is further based on kinetic and thermodynamic properties of removing the solvent mixture via spray drying.

4. The method of claim 1, wherein the pharmaceutical formulation further comprises an excipient, and wherein the excipient is included in the solution in the solvent mixture and comprises at least one of hypromellose acetate succinate, hydroxypropyl methylcellulose, polyvinylpyrrolidone, polyvinylpyrrolidone-vinyl acetate copolymer, and polymethacrylate and the solvent mixture comprises at least one of dimethylsulfoxide (DMSO), n-methyl-2-pyrrolidone (NMP), dimethylformamide (DMF), and n,n-dimethylacetamide (DMAC).

5. The method of claim 1, wherein dimethylsulfoxide (DMSO) is selected as the low volatility solvent based on the solubility of the drug being comparable in solvent blends including DMSO compared with solvent blends not including DMSO, and wherein the spray drying mass ratio is greater than a lower threshold spray drying mass ratio that is determined based on the process outlet temperature.

6. The method of claim 5, wherein the lower threshold spray drying mass ratio is 0.004 with respect to DMSO when the process outlet temperature is greater than or equal to 60° C., and wherein the lower threshold spray drying mass ratio is 0.001 with respect to DMSO when the process outlet temperature is less than 60° C.

7. The method of claim 1, wherein the process outlet temperature is less than an upper threshold process outlet temperature that is determined based on chemical and physical stability constraints of the pharmaceutical formulation.

8. The method of claim 7, wherein the upper threshold process outlet temperature is 90° C.

9. A method for identifying processing parameters for spray drying a pharmaceutical formulation from a low volatility solvent, comprising:
generating a process map relating a process outlet temperature of the spray drying to a spray drying mass ratio based on a glass transition temperature of the pharmaceutical formulation at each spray drying mass ratio; and
generating an equivalence plot on the process map where the glass transition temperature of the pharmaceutical formulation equals the process outlet temperature as a function of the spray drying mass ratio.

10. The method of claim 9, wherein generating the process map relating the process outlet temperature of the spray drying to the spray drying mass ratio based on the glass transition temperature of the pharmaceutical formulation at each spray drying mass ratio comprises:
determining a first relationship between a relative saturation of the low volatility solvent and the glass transition temperature of the pharmaceutical formulation;
determining a second relationship between the spray drying mass ratio and the relative saturation of the low volatility solvent at a given process outlet temperature of the spray drying based on the first relationship; and
determine the glass transition temperature of the pharmaceutical formulation at each spray drying mass ratio based on the second relationship.

11. The method of claim 9, further comprising:
identifying a maximum of the equivalence plot corresponding to a maximum spray drying mass ratio of the equivalence plot; and
identifying a corresponding process outlet temperature at the maximum of the equivalence plot.

12. The method of claim 11, further comprising:
selecting the maximum spray drying mass ratio and a process outlet temperature that is less than or equal to the corresponding process outlet temperature at the maximum of the equivalence plot as the processing parameters for spray drying the pharmaceutical formulation.

13. The method of claim 11, further comprising:
selecting a portion of the low volatility solvent that is lower than a highest portion of low volatility solvent based on at least one of a solubility of the pharmaceutical formulation in the low volatility solvent, a viscosity of the pharmaceutical formulation in the low volatility solvent, and a temperature sensitivity of the pharmaceutical formulation.

14. The method of claim 9, wherein the equivalence plot defines a maximum spray drying mass ratio as a function of the process outlet temperature for the spray drying.

15. A system for preparing a pharmaceutical formulation, comprising:
a solution of a drug and an excipient dissolved in a solvent blend comprising at least one low volatility solvent and at least one high volatility solvent, wherein the at least one low volatility solvent comprises at least 10% of the solvent blend; and
a spray dryer configured to receive the solution and a stream of drying gas and output a dried dispersion of the drug and the excipient as the pharmaceutical formulation, wherein a process outlet temperature of the spray dryer is selected as a function of a spray drying mass ratio of a liquid feed rate of the solution to a gas feed rate of the drying gas.

16. The system of claim 15, wherein the at least one low volatility solvent is selected from dimethylsulfoxide (DMSO), n-methyl-2-pyrrolidone (NMP), dimethylformamide (DMF), and n,n-dimethylacetamide (DMAC) and wherein at least one high volatility solvent is selected from acetone, methanol, ethanol, isopropanol, ethyl acetate, acetonitrile, tetrahydrofuran, methylene chloride, n-methyl tetrahydrofuran, n-propanol, chloroform, hexanes, acetic acid, cyclohexane, ethyl ether, and water.

17. The system of claim 15, wherein the process outlet temperature of the spray dryer is less than a glass transition temperature of the dried dispersion of the drug and the excipient.

18. The system of claim 15, wherein a relative saturation of the at least one low volatility solvent at an outlet of the spray dryer at equilibrium is between 5 and 50%.

19. The system of claim 15, wherein the spray drying mass ratio of the liquid feed rate of the solution to the gas feed rate of the drying gas is selected to maximize an amount of the drug and the excipient processed per unit of time.

20. The system of claim 15, wherein the solvent blend is selected according to a solubility of the drug and the excipient in the solvent blend and a maximum spray drying mass ratio of the liquid feed rate of the solution to the drying gas using the solvent blend.
